(19) United States
(12) Patent Application Publication
(10) Pub. No.: US 2026/0132102 A1
(43) Pub. Date: May 14, 2026

(54) UREA-SOLUTION EFFLORESCENCE DISSOLVING ADDITIVE AND ITS MANUFACTURING METHOD

(71) Applicant: Segil YUN, Eumseong-gun (KR)

(72) Inventor: Segil YUN, Eumseong-gun (KR)

(21) Appl. No.: 19/118,761

(22) PCT Filed: Aug. 29, 2023
(86) PCT No.: PCT/KR2023/012834

§ 371 Date: Apr. 4, 2025

(30) Foreign Application Priority Data
Oct. 6, 2022 (KR) 10-2022-0128199

(51) Int. Cl. C07C 273/16; C07C 275/02

(52) U.S. Cl. CPC C07C 273/16; C07C 275/02

(57) ABSTRACT

The present invention relates to a urea-solution efflorescence dissolving additive and a method for manufacturing the same, and more particularly, to a urea-solution efflorescence dissolving additive and a method for manufacturing the same, comprising: a step of manufacturing (S1) a mixture by stirring and mixing monoethanolamine, triethanolamine, methylpropylenetriol, and methylpropylene glycol; a filtering step (S2) of filtering the mixture to manufacture a filtered mixture; and a removing step (S3) of removing metal ions and impurities from the filtered mixture to manufacture an impurity-removed mixture; thereby providing a urea-solution efflorescence dissolving additive and a method for manufacturing the same, wherein the present invention is mixed with urea-solution injected into an existing SCR catalyst device for a diesel vehicle to minimize efflorescence occurring in a urea-solution nozzle and a urea-solution pump, and to clean adsorbed urea-solution crystals while promoting normal operation of the SCR to reduce carbon dioxide and NOx emissions.

### TECHNICAL FIELD

The present invention relates to a urea-solution efflorescence dissolving additive and a method for manufacturing the same, and more particularly, to a urea-solution efflorescence dissolving additive which can minimize efflorescence of urea-solution injected into an SCR (Selective Catalyst Reduction) device of a diesel vehicle and adsorption or hardening of the urea-solution on an injection nozzle, a dosing pump, or a sensor, or can dissolve and discharge hardened urea-solution crystals.

### BACKGROUND ART

Recently, as environmental pollution issues have become a major issue, eco-friendly cars that do not use fossil fuels, such as electric cars, hydrogen cars, solar cars, and hybrid cars, are actively being developed and sold in the automobile market. However, despite the emergence of such eco-friendly cars, gasoline or diesel-powered cars still dominate the automobile market, and it is expected that large diesel trucks will continue to exist for a considerable period of time.

In the case of internal combustion engine cars that use fossil fuels such as gasoline or diesel, there is a serious problem of environmental pollution due to exhaust gas. In particular, in the case of diesel cars such as buses and trucks, the emission of soot, nitrogen oxides (NOx), and fine dust including soot has become a serious problem, and each country is strictly regulating the emission of exhaust gas by establishing related regulations to solve the exhaust gas problem of diesel vehicles.

Known methods for treating exhaust gases from such diesel engines include oxidation catalyst methods and NOx catalyst methods, and DPF (Diesel Particulate Filter) methods for removing fine dust including smoke. Since diesel engines are operated in an air-excessive state in most areas, a large amount of oxygen is emitted during exhaust. Methods for reducing NOx based on the presence of such oxygen include methods for selectively decomposing NOx using SCR (Selective Catalytic Reduction) catalysts coated with copper or iron ions and ammonia as a reducing agent.

In these SCR systems, ammonia is generated using urea instead of ammonia gas as a reducing agent. The urea stored inside the urea storage tank is automatically supplied to the dosing unit in the required amount. Normally, about 4% of the fuel consumed is consumed. If the urea is left at room temperature for about 18 months, limescale precipitation occurs in the urea. In addition, although urea sprayed in exhaust gas over 300 degrees Celsius can convert to ammonia gas and remove nitrogen oxides, the temperature of the exhaust gas is generally irregular. Therefore, at temperatures between 180 and 200 degrees Celsius, the urea cannot convert to ammonia gas, and limescale-like urea crystals are formed, causing a efflorescence. This efflorescence accumulates in the urea-solution injection nozzle and urea-solution pump, gradually hindering the injection of urea solution. If left untreated, the nozzle or pump becomes completely blocked, preventing the urea-solution from being injected, which causes an overload on the DPF soot reduction device and reduces engine output.

That is, if urea-water is used for a certain period of time, efflorescence occurs in the urea-water injection nozzle and urea-water pump, which hinders urea-water injection, causing problems in terms of business and economic damage to the operation of diesel vehicles, such as reduced engine output, reduced fuel efficiency, and increased smoke emissions. In order to prevent clogging of the urea solution injection nozzle as described above, in the exhaust gas denitrification system capable of preventing clogging of the urea solution inlet passage and the injection nozzle according to Application No. 10-2010-0005110 and the urea solution supply device capable of preventing urea solution coagulation, the urea solution injection unit comprises: an air supply unit which supplies external air to the injection unit through an air inlet passage; a urea solution supply unit which supplies urea solution to the injection unit through the urea solution inlet passage; a water supply unit which supplies water to the injection unit through a water inlet passage connected to one side of the urea solution inlet passage; a flow control valve which selectively supplies water or urea solution to the injection unit through the urea solution inlet passage; and a spray unit which is respectively connected to the air supply unit and the urea solution supply unit and selectively discharges air, urea solution, and water into a reaction chamber through the spray nozzle. Including, an exhaust gas denitrification system capable of preventing clogging of the urea water inlet passage and the injection nozzle by supplying water to the injection nozzle and urea water inlet passage that become clogged during the process of continuously discharging urea water and air into the reaction chamber, and a urea water supply device capable of preventing urea water coagulation are proposed. However, if urea water crystals are already adsorbed and coagulated on the injection nozzle, etc. due to the efflorescence of urea water, a method for dissolving and washing them is not proposed.

In general, due to the above-mentioned efflorescence, hot water is repeatedly sprayed 5 to 6 times with strong water pressure to wash away the solidified and adsorbed urea crystals, but the limestone urea crystals accumulated due to the efflorescence are not effectively removed, and furthermore, they cause damage or destruction of parts. This efflorescence frequently occurs in large commercial vehicles, and individual owners or companies that own large commercial vehicles are experiencing great difficulties in vehicle maintenance.

In this regard, the present invention relates to a urea-solution efflorescence dissolving additive and a method for manufacturing the same, and more particularly, to a urea-solution efflorescence dissolving additive capable of effectively cleaning by dissolving urea-water crystals that are whitened and adsorbed on the surface of parts in an SCR device, and a method for manufacturing the same.

### DISCLOSURE

### Technical Problem

The present invention relates to a urea-solution efflorescence dissolving additive and a method for manufacturing the same, and aims to provide a urea-solution efflorescence dissolving additive capable of effectively cleaning urea-water crystals that are whitened and adsorbed on the surface of parts in an SCR device, and a method for manufacturing the same.

### Technical Solution

In order to solve the above-mentioned problem,
the present invention provides a urea-solution efflorescence dissolving additive and a method for manufacturing the same, which are manufactured through a mixture manufacturing step (S1) of stirring and mixing monoethanolamine, triethanolamine, methyl propylene triglycol, and methyl propylene glycol to manufacture a mixture;
a filtering step (S2) of filtering the mixture to manufacture a filtered mixture; and
an impurity removal step (S3) of removing metal ions and impurities from the filtered mixture to manufacture an impurity-removed mixture.

### Advantageous Effects

The present invention relates to a urea-solution efflorescence dissolving additive and a manufacturing method thereof.

Currently, there is no chemical cleaning agent capable of cleaning urea-solution crystals aggregated in an SCR device of a diesel vehicle. Therefore, in order to clean urea-solution efflorescence (crystal dissolution and cleaning performance), high-pressure water is sprayed or physical external force is applied to clean, which causes inconveniences such as damage to vehicle parts or a long cleaning time. On the other hand, the present invention mixes monoethanolamine, triethanolamine, methyl propylene triglycol, and methyl propylene glycol according to the mixing ratio set forth by the present invention, manufactures a chemical mixture, and mixes it with urea-solution sprayed into an existing diesel vehicle SCR catalyst device, thereby effectively cleaning efflorescence of a urea-solution nozzle and a urea-solution pump, while promoting normal operation of the SCR device. As a result, it is possible to reduce carbon dioxide and NOx and improve fuel efficiency in terms of the environment. In general, when requesting urea-solution crystal removal and cleaning to mechanics, it takes about 5 to 6 hours of work time, resulting in labor costs of about 1 million to 1.5 million won. When the mixture of this invention is mixed with urea-water and used, it has the advantage of being able to effectively reduce costs and time required for cleaning, as the urea-water crystals adsorbed and coagulated inside the SCR device can be easily removed by dissolving them.

### BRIEF DESCRIPTION OF THE DRAWINGS

FIG. 1 illustrates a flow chart of a method for manufacturing a detergent additive for efflorescence of elements.

### DETAILED DESCRIPTION

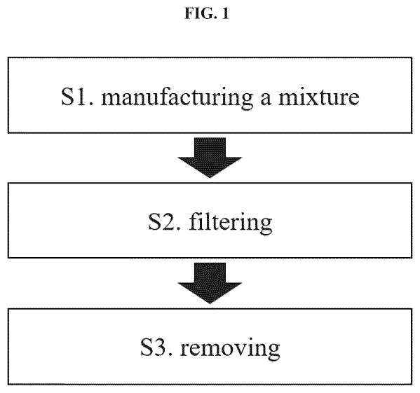

Hereinafter, the attached FIG. 1 will be referred to for a more detailed explanation.

Prior to this, the terms or words used in this specification and claims should not be interpreted as limited to their usual or dictionary meanings, and should be interpreted as meanings and concepts that conform to the technical idea of the present invention based on the principle that the inventor can appropriately define the concept of the term in order to explain his or her own invention in the best way.

Therefore, some embodiments described in this specification and the configurations illustrated in the drawings are the preferred embodiments of the present invention, and do not represent all of the technical ideas of the present invention. Therefore, it should be understood that there may be various equivalents and modified examples that can be replaced at the time of this application.

The present invention relates to a urea-solution efflorescence dissolving additive and a method for manufacturing the same, and more particularly,
to a urea-solution efflorescence dissolving additive and a method for manufacturing the same, which are manufactured through a mixture manufacturing step (S1) of mixing and stirring monoethanolamine, triethanolamine, methyl propylene triglycol, and methyl propylene glycol to manufacture a mixture;
a filtering step (S2) of filtering the mixture to manufacture a filtered mixture; and

an impurity removal step (S3) of removing metal ions and impurities from the filtered mixture to manufacture an impurity-removed mixture.

More specifically,
the mixture preparation step (S1) is a step of manufacturing a mixture by stirring 40 to 45 wt % of monoethanolamine, 10 to 15 wt % of triethanolamine, 30 to 35 wt % of methylpropylene triglycol, and 10 to 15 wt % of methylpropylene glycol at 10 to 20 degrees Celsius for 30 to 60 minutes, as shown in [Table 1] below.

As described above, the mixture is stirred at 10 to 20 degrees Celsius for 30 to 60 minutes, but triethanolamine in the mixture has a problem in that it is difficult to mix with other substances at low temperatures due to its high viscosity due to its physical properties. Therefore, in order to smoothly mix with other components, a process of stirring at the optimal temperature of 10 to 20 degrees Celsius for 30 to 60 minutes is necessary, and when the mixture is stirred at less than 10 degrees Celsius for less than 30 minutes, there was a problem in that it was difficult to mix evenly with other substances due to the viscosity.

TABLE 1

| Composition | Weight (%) |
|---|---|
| Monoethanolamine | 40~45% by weight, |
| Triethanolamine | 10~15% by weight, |
| Methylpropylenetriglycol | 30~35% by weight |
| Methyl propylene glycol | 10~15% by weight, |

The monoethanolamine according to some embodiments has a chemical formula of H₂NCH₂CH₂OH and has excellent oil-removing ability and prevents re-contamination, but it has strong viscosity and has the property of causing an unpleasant odor. 40 to 45 wt % of monoethanolamine in the mixture can effectively decompose high pH urea crystals (lime, pH 12.1), but when monoethanolamine is mixed in an amount less than 40 wt %, the limestone decomposition time is about 10 to 15% longer, so when the consumption of urea-water is taken into consideration, the cleaning effect as an additive is halved, and when monoethanolamine is mixed in an amount exceeding 45 wt %, there is a problem that the urea-water nozzle and surrounding metal parts may turn black and corrode.

The triethanolamine according to some embodiments has the chemical formula C6H15NO3 and is not a toxic or hazardous substance, has a surfactant effect, and has the characteristics of a flash point of 179 degrees Celsius, which is higher than the flash point of monoethanolamine, which is 85 degrees Celsius. In particular, as described above, when triethanolamine was used as a single component, it did not mix smoothly with urea-water due to its high viscosity, but when used mixed with the monoethanolamine, the solubility in water improved, and the flash point was improved by mixing, so that the decomposition rate of urea-water crystals (lime) at high temperatures was faster than when monoethanolamine was used alone, and the reaction time was shortened. However, when less than 10% by weight of triethanolamine is mixed in the mixture, the cleaning effect is present, but the limestone decomposition speed is delayed by about 15%, and when more than 15% by weight of triethanolamine is mixed in the mixture, it causes the accumulation of limestone dissolved in the urea-solution filter, and since monoethanolamine is 40-45%, when more than 15% of triethanolamine is mixed, the total amine series mixing ratio exceeds 60%, which causes an increase in the ammonia pH, resulting in blackening of the urea-solution nozzle and related parts.

Furthermore, since the viscosity of monoethanolamine is lower than that of triethanolamine, it mixes well with urea-water, and since monoethanolamine has a pH of 12.1, which is the highest among the amines, it has the best crystal dissolution at room temperature, but has a low flash point (85 degrees Celsius), so the cleaning effect is reduced at temperatures above 180 degrees Celsius. To improve this, triethanolamine (179 degrees), which has high viscosity and a flash point more than twice as high, was mixed, and since the mixing efficiency with urea-water is considered due to its high viscosity, monoethanolamine and triethanolamine were mixed in a ratio of 4:1, which can effectively promote the dissolution of urea-water crystals at room temperature and high temperatures, and since it has the surfactant effect of triethanolamine, the cleaning effect can be improved.

Monoethanolamine and triethanolamine are components that play a direct role in the dissolution of urea-water crystals. However, when triethanolamine was used alone, the decomposition of the crystals could be confirmed after about 48 hours due to its high viscosity, and when monoethanolamine was used alone, the crystals were dissolved for only about 24 hours. In other words, when monoethanolamine was used alone, the decomposition speed of urea-water crystals was observed as well as when monoethanolamine and triethanolamine were mixed, but there was a problem that the cleaning power was halved before the additive reached the urea-water nozzle because the flash point of monoethanolamine was low. In order to solve the above-mentioned problem, monoethanolamine (flash point 85 degrees Celsius) with a low flash point and triethanolamine (flash point 179 degrees Celsius) with a high flash point were mixed and used.

In addition, the ethanolamine series according to some embodiments not only has secured cleaning power but is also environmentally friendly, whereas triethylamine or isopropylamine, which were previously used for cleaning metal parts, etc., have low flash points and are also classified as toxic or hazardous substances, so the monoethanolamine according to some embodiments, which is more environmentally friendly and safe, was used in the production of the mixture.

The methyl propylene triglycol according to some embodiments is CH₃(OCH₂CHCH₃)₃OH, and is an organic synthetic raw material used as a solvent, dispersant, and diluent. It has the characteristics of promoting the dissolution of aggregated carbon and solid fine particles and minimizing secondary aggregation, helping the smooth dilution of monoethanolamine and triethanolamine, and dispersing the dissolved urea crystals and preventing corrosion of metal parts to minimize the re-aggregation of urea crystals in urea nozzles, etc. In addition, since urea crystals are generated due to the inconsistent temperature of exhaust gas, methyl propylene triglycol having a boiling point of 242 degrees Celsius was mixed in to suppress the generation of crystals below 200 degrees Celsius and induce dispersion, and to form a film in the nozzle and SCR at high temperatures.

When methylpropylene triglycol is mixed in the mixture in an amount of less than 30 wt %, the ratio of monoethanolamine and triethanolamine increases to 60 wt % or more, which enhances the cleaning power, but increases the risk of discoloration and corrosion of parts. In addition, when methylpropylene triglycol is mixed in an amount exceeding 35 wt %, the total mixing ratio of monoethanolamine and triethanolamine decreases to 60% or less, which weakens the cleaning effect. As a result, the coagulation and dispersion effect of the urea crystals by methylpropylene triglycol is not enhanced.

The methyl propylene glycol according to some embodiments has the chemical formula CH₃—OCH₂CHCH₃—OH and plays a similar role to methyl propylene triglycol. It is an organic synthetic raw material used as a solvent, dispersant, and diluent, and has an excellent dissolving effect for coagulated carbon and solid fine particles and is very effective in preventing secondary coagulation. That is, like methyl propylene triglycol, methyl propylene glycol helps smooth dilution of monoethanolamine and triethanolamine, and disperses dissolved urea crystals and prevents corrosion of metal parts to minimize re-coagulation of urea crystals in urea nozzles, etc. Methyl propylene glycol has a boiling point of 121 degrees Celsius and a pour point of-95 degrees Celsius. Therefore, when urea crystals generated in exhaust gas at 200 degrees Celsius accumulate in the device, it can induce dispersion at low temperatures (room temperature) and promote film formation in the nozzle and SCR. When methylpropylene triglycol is mixed in the mixture at less than 10 wt % (i.e., when the total ratio of methylpropylene glycol and methylpropylene triglycol is less than 40 wt %), the ratio of monoethanolamine and triethanolamine increases to 60% or more, which increases the risk of discoloration and corrosion. In addition, emulsifiers such as methylpropylene triglycol do not play a significant role in the dissolution of urea crystals, but in order to minimize the re-agglomeration of urea crystals dissolved by monoethanolamine and triethanolamine at the unstable exhaust gas temperature with a deviation of 180 to 300 degrees, methylpropylene glycol and methylpropylene triglycol were mixed and used, and the mixed use at a ratio of 4 to 1 improved the dissolution of additives and urea crystals and the decomposition effect of urea crystals.

Ethanolamine series according to some embodiments can play a role in decomposing and dissolving urea crystals (lime), and methyl propylene triglycol and methyl propylene glycol have an emulsifying effect that prevents re-aggregation of decomposed or dissolved urea crystals. Since a mixture containing water-soluble components such as methyl propylene triglycol and methyl propylene glycol must be easily mixed with urea-water at room temperature and high temperature, methyl propylene triglycol with a high boiling point and methyl propylene glycol with a low pour point were mixed.

The filtration step (S2) according to some embodiments is a step for filtering dust and foreign substances using a microfilter having a filtration particle size of 1 micron or less to produce a filter mixture, and the impurity removal step (S3) according to some embodiments is a step for producing an impurity-removed mixture by removing impurities and metal ions from the filter mixture.

The mixture created through the above-mentioned steps can be effectively dissolved by mixing it with urea-water without the need for separate equipment and without the detachment or installation of parts, and discharged together with exhaust gas by accumulating white matter (lime) in the urea-water nozzle and pump, thereby relieving DPF overload, improving performance, enhancing engine output, helping vehicle operation, and reducing carbon dioxide and NOX emissions through normalization of SCR.

As examples,

commercial vehicle 1 (Iveco, mileage 580,000 km) and large commercial vehicle 2 (Iveco, mileage 110,000 km) were selected as vehicles that did not use any additives at all, and commercial vehicles 1 and 2 had similar symptoms with reduced output and nozzles and pumps that did not operate normally. The urea-solution efflorescence dissolving additive of our institute was mixed with 500 ml of additive in 10 liters of urea-water and injected into the above-mentioned vehicles 1 and 2 for testing.

TABLE 2

| | Monoethanolamine | | Triethanolamine | | Methylpropylenetriglycol | | Methylpropylene glycol | |
|---|---|---|---|---|---|---|---|---|
| Mixing ratio | 40~45% | by weight | 10~15% | by weight | 30~35% | by weight | 10~15% | by weight |
| Experiment 1 | 35% | by weight | 15% | by weight | 35% | by weight | 15% | by weight |
| Experiment 2 | 45% | by weight | 15% | by weight | 30% | by weight | 10% | by weight |

TABLE 3

| | Commercial vehicle 1 | Commercial vehicle 2 |
|---|---|---|
| | (mileage 580,000) | (mileage 110,000) |
| Experiment 1 | About 2,000 km | About 2,000 km |
| Experiment 2 | About 1,000 km | About 1,000 km |

| | |
|---|---|
| General products | No existing products |
| Notes | *Driving distance when cleaning effect |
| | is present after adding additives |

In Experiment 1, both vehicles drove 2,000 km over approximately 3 days before the engine output returned to normal and the carbon dioxide and NOx levels returned to normal. In Experiment 2, after driving 1,000 km over approximately 2 days before the engine output returned to normal and the carbon dioxide and NOx levels returned to normal. In other words, Experiment 2 dissolved the efflorescence (lime crystals) more efficiently, and the carbon dioxide and NOx levels emitted from the exhaust gas of Experiment 2 were 1 to 2 lower than those of Experiment 1.

The accumulated amount and accumulated time of urea crystals are closely related to the driving environment of commercial vehicles and have little to do with the accumulated driving distance. That is, in the case of commercial vehicles, the urea nozzle becomes clogged after about 100,000 km for vehicles that drive long distances at high speeds, and after about 20,000 km for vehicles that drive frequently for short distances, making driving impossible due to reduced output. As shown in the table above, the accumulated amount of urea crystals in the device of a vehicle is similar regardless of the driving distance. Therefore, since the decomposition speed of limestone under driving conditions after additive injection is fast, there is almost no difference in the decomposition speed between vehicles with high and low driving distances. As shown in Experiment 2 of Tables 2 and 3, the higher the weight % of the ethanolamine series, the faster the dissolution speed of the aggregated urea crystals.

The effects of the efflorescence-prevention cleaning additives of the element water manufactured in Experiments 1 and 2 were measured and shown in Table 4.

Experiments 1 and 2 were conducted on large commercial vehicles from the same manufacturer, No. 1 (Man Truck, 350,000 km) and No. 2 (Man Truck, 440,000 km), which did not use any efflorescence detergent or any other additives. The two vehicles had reduced output and clogged urea-water nozzles, making normal operation difficult. The nozzles of the two vehicles, which had similar symptoms, were separated, placed in detergent, and stored separately in a sealed container.

TABLE 4

| | Separation nozzle for a vehicle | Separation nozzle of a vehicle |
|---|---|---|
| | that has driven 350,000 km | driven 440,000 km |
| Sealing and storage time after | About 18 hours | About 18 hours |
| adding additives (mixing ratio | | |
| of Experiment 1) | | |
| Sealing and storage time after | About 12 hours | About 12 hours |
| adding additives (mixing ratio | | |
| of Experiment 2) | | |

The holes in the element water nozzles separated from both vehicles were completely dissolved in the additive and removed after 12 hours, and no secondary problems such as damage to parts, hardening or corrosion of parts occurred after the removal of the efflorescence (lime crystals).

As shown in Table 4, it was confirmed that the efflorescence (lime crystals) was clearly removed by the effect of the efflorescence cleaning additive of the urea-water of Experiments 1 and 2. It can be seen that the mixing standards do not have a large difference in the performance of efflorescence (lime crystals) in both Experiments 1 and 2, and it can be seen from all the experimental results in Tables 2 to 4 that it shows excellent performance in removing efflorescence at room temperature and high temperature. Therefore, it was confirmed that the urea-water efflorescence cleaning agent according to the present invention can be removed more easily and efficiently than the existing efflorescence cleaning maintenance process when used by maintenance companies, and it seems that many improvements in work are possible as the difficulty of maintenance is lowered. In addition, since the detergent and wastewater generated through maintenance are composed of pure ammonia, a water-soluble detergent, and purified water, they can be treated, so it is expected that the burden on environmental issues will be reduced.

As described above, the present invention has been described by specific matters such as specific components, limited embodiments, and drawings, but these are only provided to help a more general understanding of the present invention, and the present invention is not limited to the above-mentioned embodiments, and those with common knowledge in the field to which the present invention pertains can make various modifications and variations from this description.

Therefore, the idea of the present invention should not be limited to the described embodiments, and not only the following patent claims, but also all things that are equivalent or equivalent to the claims are considered to belong to the scope of the present invention.

1. A method for manufacturing a urea-solution efflorescence dissolving additive, the method comprising:
manufacturing (S1) a mixture by stirring and mixing monoethanolamine, triethanolamine, methylpropylenetriol, and methylpropylene glycol;
filtering (S2) the mixture to manufacture a filtered mixture; and
removing (S3) metal ions and impurities from the filtered mixture to manufacture an impurity-removed mixture.

2. The method of claim 1, wherein the manufacturing (S1) comprises preparing the mixture by mixing 40 to 45 wt % of monoethanolamine, 10 to 15 wt % of triethanolamine, 30 to 35 wt % of methylpropylenetriol, and 10 to 15 wt % of methylpropylene glycol.

3. The method of claim 1, wherein the manufacturing (S1) comprises
manufacturing the mixture by mixing monoethanolamine, triethanolamine, methylpropylenetriol, and methylpropylene glycol, and stirring the mixture at a temperature of 10 to 20° C. for 30 to 60 minutes.

4. The method of claim 1, wherein the filtering (S2) comprises
manufacturing the filtered mixture by filtering dust and foreign substances from the mixture using a microfilter having a filtration particle size of 1 micron or less.

5. A cleaning additive for preventing urea-solution efflorescence, manufactured by the method according to claim 1.
